(19) United States
(12) Patent Application Publication
(10) Pub. No.: US 2006/0153843 A1
(43) Pub. Date: Jul. 13, 2006

(54) Method of immunomodulation

(75) Inventors: Masato Kato, Hawthorne (AU); David Munster, Holland Park (AU); Derek Hart, Dutton Park (AU)

Correspondence Address: SCULLY SCOTT MURPHY & PRESSER, PC, 400 GARDEN CITY PLAZA, SUITE 300, GARDEN CITY, NY, 11530, US

(73) Assignee: The Corporation of The Trustees of The Sisters of Mercy in Queensland of South Brisbane, Queensland (AU)

(21) Appl. No.: 10/524,716

(22) PCT Filed: Aug. 15, 2003
(86) PCT No.: PCT/AU03/01038

§ 371 Date: Feb. 16, 2006

(30) Foreign Application Priority Data
Aug. 15, 2002 (AU) 2002950779

(51) Int. Cl. A61K 39/395

(52) U.S. Cl. 424/144.1

(57) ABSTRACT

The present invention relates generally to a method for modulating the activity of cells of the immune system, including stimulator and responder cells and to agents useful therefor. More particularly, the present invention relates to a method for preventing or down-regulating one or more functional activities of stimulator and responder cells such as, respectively, antigen-presenting cells and lymphocytes inter alia. The present invention further provides antibodies, which interact specifically with epitopes present on the surface of antigen-presenting cells and lymphocytes, resulting in depletion, down-regulation or destruction of targeted antigen-presenting cells and lymphocytes in vivo or in vitro. The instant invention further provides a method for modulating an immune response in a subject and, in particular, for down-regulating the immuno-activity of an allogeneic immuno-competent graft and/or the immune response of a recipient of a solid organ transplant. The ability to modulate stimulator and responder cell immuno-activity may be useful, inter alia, in a range of immuno-therapeutic and immuno-prophylactic treatments that benefit from immune suppression.

### BACKGROUND OF THE INVENTION

1. Field of the Invention

The present invention relates generally to a method for modulating the activity of cells of the immune system, including stimulator and responder cells and to agents useful therefor. More particularly, the present invention relates to a method for preventing or down-regulating one or more functional activities of stimulator and responder cells such as, respectively, antigen-presenting cells and lymphocytes inter alia. The present invention further provides antibodies, which interact specifically with epitopes present on the surface of antigen-presenting cells and lymphocytes, resulting in depletion, down-regulation or destruction of targeted antigen-presenting cells and lymphocytes in vivo or in vitro. The instant invention further provides a method for modulating an immune response in a subject and, in particular, for down-regulating the immuno-activity of an allogeneic immuno-competent graft and/or the immune response of a recipient of a solid organ transplant. The ability to modulate stimulator and responder cell immuno-activity may be useful, inter alia, in a range of immuno-therapeutic and immuno-prophylactic treatments that benefit from immune suppression.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Allogeneic transplantation involves the transfer of material from a host to a recipient. In this process, many foreign antigens are introduced into a host and an immune response results when these foreign antigens are detected by the host's immune system. Initially, an immune response involves interactions between the antigen and antigen-presenting cells (APC) such as dendritic cells: potent cellular activators of primary immune responses (Hart, Blood 90:3245-3287, 1997). Immature myeloid dendritic cells (DC) in non-lymphoid organs react to, endocytose and process antigens and migrate via blood and lymph to T cell areas of lymphoid organs. Here, the mature cells present foreign peptide complexed to MHC Class II to T cells and deliver unique signals for T-cell activation (immuno-stimulation). They also stimulate B lymphocytes and NK cells. DC undergo differentiation/activation during this process, lose their antigen-capturing capacity and become mature, immuno-stimulatory DC that trigger naive T-cells recirculating through the lymphoid organs.

Following allogeneic transplantation, interstitial donor DC in heart and kidney contribute to (direct) recipient T-lymphocyte sensitization to all antigens but recipient DC, after migrating into the donor tissue, can also stimulate (indirect) alloantigen sensitization of recipient T-lymphocytes. Depletion of heart and kidney and pancreatic islet DC appears to prolong allograft survival. Interestingly, during liver transplantation, donor leucocytes, which may include non-activated dendritic cells, appear to generate allogeneic tolerance. DC are also predicted to contribute to both acute and chronic Graft Versus Host Disease (GVHD), the major life threatening complication of allogeneic bone marrow transplantation (BMT). Blood DC counts change during acute GVHD. Recent evidence from a mouse model suggests that host APC, including DC, contribute to the acute GVHD. DC may in certain circumstance prevent acute GVHD.

As part of the differentiation/activation process, DC up-regulate certain relatively selectively expressed cell surface molecules such as the CMRF-44 and CD83 antigens. CD83 is a type 1 membrane protein of the immunoglobulin super-family. It is expressed on the surface of activated DC and B-cells and, at low levels, on mitogen and phorbol myristate acetate activated T-cells (Zhou et al., J Immunol 149:735, 1992; Kozlow et al., Blood 81:454, 1993). A soluble form of CD83 is also detectable in normal serum and is released from cell lines and monocyte-derived DC (MoDC) (Armitage et al., In: Leucocyte Typing VI. T. Kisimoto, ed. Garland Publishing Inc, New York, p. 593, 1996a; Hock et al., Int Immunol 13:959, 2001).

The function of CD83 is not known, although recent data from CD83-gene deleted mice suggest that its expression on thymic epithelium contributes to CD4 T-lymphocyte development (Fujimoto et al., Cell 108:755, 2002. Cramer et al. (Int Immunol 12:1347, 2000) have suggested that a ligand for CD83 is expressed on murine B-cells. In contrast, Scholler et al. (J Immunol 166:3865, 2001) recently claimed that human CD83 is a sialic acid binding Ig-like lectin adhesion receptor, the counter-receptor for which is a 72 kDa protein expressed on monocytes and a subset of activated or stressed T-cells (Scholler et al., 2001, supra). Curiously, DC and CD4⁺ T-lymphocytes from CD83^{-/-} mice functioned normally in the allogeneic mixed leucocyte reaction (MLR) and in other in vitro assays, although in vivo B-cell function was altered due to the reduced numbers of CD4⁺ T-cells.

Monoclonal antibodies (mAb) which act at the level of the responder T-lymphocyte have been investigated as therapeutic immuno-suppression agents in allogeneic transplantation. Attempts to interfere with the interaction of the responder T-lymphocyte and an APC have focused on antibodies directed against the co-stimulator molecules CD40, CD80 and CD86 or their ligands. The role of CD83 in human DC-lymphocyte interactions has also been examined experimentally. It was reported that polyclonal rabbit anti-CD83 (RA83) blocks the proliferative response of human peripheral blood mononuclear cells (PBMC) to phytohaemagglutinin, to the recall antigen tetanus toxoid (TT), and to allogeneic stimulators, although murine mAbs failed to have a significant effect (Armitage et al., In: Leucocyte Typing VI. T Kisimoto, ed. Garland Publishing Inc., New York, p. 595, 1996b; Zhou et al., J Immunol 154:3821, 1995). It was also reported that RAS3 inhibited the B-cell proliferative response of T-cell depleted PBMC to CD40L, and that it blocked CD40L +IL10 induced antibody synthesis.

In contrast, it has been reported that a murine CD83 fusion protein weakly inhibits the proliferative response of splenocytes (from DO11.10 TCR transgenic mice) to antigen, and that antigen-induced IL-2 expression is reduced by the murine CD83 fusion protein by up to 56% in this model (Cramer et al., 2000, supra). Also, Lechmann et al. (Lechmann et al., J Exp Med 194:1813, 2001) reported that ligation of human DC with synthetic CD83 extracellular domain blocks the MLR and an antigen specific T-cell response (Lechmann et al., 2001, supra). These results are difficult to reconcile with the above-mentioned finding that CD83^{-/-} DC stimulate a normal allogeneic MLR.

Theoretically, antibodies directed at DC administered to the recipient of a solid organ graft would deplete donor DC (i.e. direct alloantigen presentation), as well as recipient DC (indirect alloantigen presentation) in the graft and/or in draining lymph ducts and lymph nodes. Other donor leucocytes, including certain DC preparations administered in a tolerogenic state, may have immunomodulatory capacity. DC depletion therapy might then be ceased after a short period, allowing tolerance to emerge. Depleting recipient DC for varying time periods may be more efficacious than disrupting co-stimulator pathways. Investigation of this concept has been delayed, however, by the absence of suitable DC reagents. Given the importance of DC stimulator cells and T-lymphocyte effectors in the overall immuno-potential of an individual, there is a need to identify additional more efficacious agents, which can advantageously facilitate modulation of stimulator and responder cell activity.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the determination that a cell-surface activation molecule may act as a target for agents, the binding of which, results in disablement and/or eventual destruction of the cell. In particular, it has been shown that antibodies to the cell-surface activation molecule CD83 are capable of initiating lysis of both stimulator cells such as antigen presenting cells (APC), and responder cells such as T- and B-lymphocytes. More particularly, CD83 antibody is capable of acting as an immuno-suppressive agent, by effecting the eventual destruction of APC and T-cells expressing CD83. This may also be regarded as a down-regulation of APC. Thus, the present invention provides reagents useful for the disablement of activated stimulator APC and activated responder cells such as T-cells. It further provides a method for the suppression of an immune response useful inter alia for the reduction or prevention of allogeneic graft rejections, graft versus host disease, and the amelioration of certain auto-immune inflammatory interactions, such as rheumatoid arthritis.

The present invention, therefore, contemplates a method for modulating the immuno-activity of a stimulator cell and a responder cell by contacting said stimulator and responder cells with an effective amount of an agent which couples, binds or otherwise associates with a cell-surface activation molecule and in turn prevents, inhibits or otherwise down-regulates one or more functional activities of the said cells.

Generally, the stimulator cell is an APC and, more preferably, a dendritic cell (DC), and the responder cell is a lymphocyte and, more preferably, a cell expressing a T-cell receptor. Moreover, the stimulator and responder cells are preferably activated stimulator and responder cells.

In a preferred embodiment, the agent comprises a polyclonal or monoclonal antibody (Ab) such as, for example, CD83Ab, or a derivative, fragment, homolog, analog or chemical equivalent or mimetic thereof and the cell-surface activation molecule is a molecule or a derivative, fragment, homolog, analog or chemical equivalent or mimetic thereof, expressed on the surface of a DC and/or a T-cell, and which interacts with CD83 Ab.

The present invention is further directed to a method for modulating an immune response in a subject by administering to the subject an effective amount of an agent which couples, binds or otherwise associates with an activated stimulator cell's and an activated responder cell's surface activation molecule (e.g. a DC and/or T-cell surface molecule which interacts with CD83 Ab) which in turn prevents, inhibits or otherwise down-regulates one or more functional activities of the activated cells.

The agent of the present invention may also be used to down-regulate the immuno-activity of an immuno-competent graft such as a bone marrow graft or to deplete residual recipient DC which might trigger acute graft versus host disease.

Another aspect of the present invention contemplates a method for the prophylactic and/or therapeutic treatment of a condition characterized by the aberrant, unwanted or otherwise inappropriate immuno-activity of an immuno-competent graft by contacting the graft with an effective amount of the agent or a derivative, homolog, analog, chemical equivalent or mimetic thereof which prevents, inhibits or otherwise down-regulates the inappropriate immuno-activity of the graft.

The present invention further extends to pharmaceutical compositions and formulations comprising the agent for-use in conjunction with the instant methods, and to the use of such agents in the manufacture of a pharmaceutical composition or formulation.

### BRIEF DESCRIPTION OF THE FIGURES

FIG. 1 shows graphical representations indicating cell surface CD83 and CD86 expression on MoDC and CD11c⁺ blood DC (Sorg et al., Pathology 29:294, 1997). (A) CD83 and (B) CD86 expression of 48 hr cultured CD11c⁺ blood DC in GM-CSF and IL-3 (representative example of 3 experiments). (C) Time course of CD83 and CD86 expression for LPS activated MoDC and for (D) CD11c⁺ blood DC cultured in GM-CSF and IL-3 (MFI expressed as percentage of maxima. Representative examples of 2 (C) and 3 (D) experiments).

FIG. 2 shows graphical representations indicating expression of cytoplasmic and soluble CD83 by DC. Cytoplasmic staining for CD83 in (A) iMoDC and (B) LPS activated MoDC (representative of n=3 experiments). (C) Time course of soluble CD83 secretion by MoDC exposed to LPS at 0 hr (n=2 experiments), and by freshly isolated blood DC cultured in GM-CSF and IL-3 (n=3 experiments).

FIG. 3 shows graphical representations indicating effect on MoDC CD83 expression of coculture with allogeneic T-cells. (A) Percentage CD83⁺ MoDC at 0 hr and after 48 hr of culture with a 20-fold excess of allogeneic T-cells (n=6 independent experiments shown). (B) CD83 expression (MFI—X-axis) for MoDC cultured for 48 hr with 20-fold excess allogeneic T-cells or with LPS at 1 μg/ml (representative example of n=3 experiments).

FIG. 4 shows graphical representations indicating blockade of MLR with RA83. Proliferative response (cpm) of (A) rosette purified PBMC (ER⁺) or of (B) same cells further purified by immunomagnetic depletion (10⁵/well) versus number of allogeneic iMoDC/well, in the presence of 5 μg/ml RA83 or RAneg, or no antibody (representative example of n=6 experiments). (C) Failure of blockade of MLR by immunoreactive F_{ab} fragments of RA83 (n=1 experiment). (D) Effect of RA83 and number of added NK-cells on the proliferative response of sort-purified T-cells to allogeneic MoDC. The CD16 function blocking mAb 3G8 reversed the effect of the added NK-cells (representative example of n=2 experiments).

FIG. 5 shows graphical representations indicating effect of prior fixation of iMoDC on RA83 mediated blockade. Proliferative response of ER⁺ cells to (A) fresh and to (B) paraformaldehyde fixed allogeneic MoDC (representative example of n=2 experiments).

FIG. 6 shows graphical representations indicating CD83 expression by T-cells in the allogeneic MLR. (A) Time course of percentage CD83⁺, CD25⁺, and CD83⁺ CD25⁺ T-cells stimulated by allogeneic iMoDC (representative example of n=2 experiments). Dot-plots of (B, E) forward and side light scatter, and (C, D, F, G) fluorescence intensity after (B, C) 0 hr, (D) 3 hr, and (E, F, G) 96 hr of culture. (C, D, F) gated on region shown in (B) and on CD3-FITC⁺ cells. (G) gated on CD3-FITC⁺ cells in the high forward scatter region shown in (E) which excludes the resting lymphoid cells.

FIG. 7 shows a graphical representation indicating the effect on MLR of delayed addition of RA83. Proliferative response (cpm) of 10⁵ ER⁺ cells per well to 2500 allogeneic iMoDC added at time 0 hr. RA83, RAneg or medium only were added at the times shown (representative example of n=2 experiments).

FIG. 8 shows a graphical representation indicating the effect of RA83 on NK-cell mediated lysis of T-cell blasts. T-cell blasts and NK-cells were sort purified from a 65 hr MLR consisting of ER⁺ cells and allogeneic iMoDC (20:1 ratio). The T-cell blasts were labelled with ⁵¹CrO₄ and co-cultured for 4 hr with the NK-cells at the ratios shown, with either RA83 or RAneg. T-cell lysis was measured as release of ⁵¹Cr into the medium (100%=⁵¹Cr released by Triton X-100) (representative example of n=3 experiments).

FIG. 9 shows graphical representations indicating that RA83 also depletes activated DC in the MLR (A) and (B) are flow cytometer dot-plots showing that activated blood DC (CMF-56⁺, CD14/19⁻ cells—lower right quadrant) in PBMC from 2 donors co-cultured for 46 hours were 89% depleted in the presence of (A) CD83 antibody (RA83) relative to (B) negative control antibody (RAneg).

FIG. 10 shows graphical representations indicating that RA83, but not RAneg, plus NK-cells purified from an allogeneic MLR can lyse CD83⁺ T-cell blasts (A) and CD83⁺ activated MoDC (B), but not CD83⁻ immature MoDC (D). (C) is a positive control showing that the NK-cells are functional.

### DETAILED DESCRIPTION OF THE PREFERRED EMBODIMENTS

The present invention is predicated in part on the observation that the proliferation of a lymphocyte such as, for example, a T-cell-receptor-expressing lymphocyte, can be suppressed via the specific targeting of an activation antigen with an effective down-regulatory agent. The present invention is further predicated on the observation that the activity of an APC such as, for example, a dendritic cell, can also be suppressed via the specific targeting of the same activation antigen with the said down-regulatory agent. The targeted lymphocyte and/or APC is/are thereby disabled or destroyed, leading to the potentially negative effects of such cells being reduced or prevented.

It must be noted that, as used in the subject specification, the singular forms “a”, “an” and “the” include plural aspects unless the context clearly dictates otherwise. Thus, for example, reference to an “antigen presenting cell” includes a single antigen presenting cell, as well as two or more antigen presenting cells; reference to a “dendritic cell” includes a single dendritic cell, as well as two or more dendritic cells; and so forth.

The identification of the capability to specifically down-regulate targeted lymphocytes and APCs enables applications as diverse as removing or reducing the rejection difficulties caused by host versus graft and graft versus host incompatibility, and ameliorating a range of auto-immune inflammatory reactions characterized by unwanted immune responses such as, for example, rheumatoid arthritis. Moreover, because stimulator cells such as APCs as well as responder cells such as lymphocytes are both able to be targeted, and their respective activities affected, more effective and efficient immuno-modulatory agents may be provided.

Accordingly, one aspect of the present invention contemplates a method for modulating the immuno-activity of a stimulator cell and a responder cell by contacting said stimulator and responder cells with an effective amount of an agent which couples, binds or otherwise associates with a cell-surface activation molecule and in turn prevents, inhibits or otherwise down-regulates one or more functional activities of the said cells.

Generally, “stimulator cells” are cells the function of which is to up-regulate one or more functional capabilities of a cell with which it interacts, such as via effecting their further proliferation and/or differentiation into functionally activated cells. Stimulator cells of the immune system include, for example, APCs. “Responder cells” are those which become functionally active in response to a signal such as, but not limited to, detection of an antigen.

In the context of the present invention, the stimulator cell is an antigen-presenting cell (APC) and, more preferably, a dendritic cell (DC), and the responder cell is a lymphocyte and, more preferably, a cell expressing a T-cell receptor.

An “antigen-presenting cell” or “antigen-presenting cells” or their abbreviations “APC” or “APCs”, as used herein, refer to a cell or cells capable of endocytotic adsorption, processing and presenting of an antigen. The term “antigen presenting” means the display of antigen as peptide fragments bound to MHC molecules, on the cell surface. Many different kinds of cells may function as APCs including, for example, macrophages, B cells, follicular DC and DC.

An “antigen” is any organic or inorganic molecule capable of stimulating an immune response. The term “antigen” as used herein extends to any molecule such as, but not limited, to a peptide, polypeptide, protein, nucleic acid molecule, carbohydrate molecule, organic or inorganic molecule capable of stimulating an immune response.

“Lymphocytes” may be T-lymphocytes or B-lymphocytes. Preferred lymphocytes of the present invention include cells whose function is to detect and/or distinguish different type of antigen or to cause the lysis of target cells expressing a particular antigen. In the context of the present invention, a particularly useful lymphocyte is a T-cell-receptor-expressing lymphocyte, generated in the thymus. The terms “T-cell-receptor-expressing cell” or “T-cell-receptor-expressing cells”, or their abbreviations “TRE” and “TREs”, as used herein, refer to any thymus-derived cell capable of detecting an antigen and effecting a cell-mediated and/or a humoral immune, response. Preferred TREs are T-lymphocytes. The terms “T-cell” and “T-lymphocyte” are used throughout synonymously. The present invention extends, however, to encompass embodiments wherein the responder cell is a B-lymphocyte.

One particularly useful APC in the context of the present invention is a DC. DC are a population of widely distributed leucocytes that are highly specialized in antigen presentation via MEC II antigen and peptide complexes. They are the principal activators of resting T cells in vitro and a major source of immunogenic epitopes for specific T cell clones following the detection of an antigen in vivo or in vitro. As used herein, the term “dendritic cell” or “dendritic cells” (DC) refers to a dendritic cell or cells in its broadest context and includes any DC that is capable of antigen presentation. The term includes all DC that initiate an immune response and/or present an antigen to T-lymphocytes and/or provide T-cells with any other activation signal required for stimulation of an immune response.

Reference herein to “DC” should be read as including reference to cells exhibiting dendritic cell morphology, phenotype or functional activity and to mutants or variants thereof and to precursor cells of DC. The morphological features of dendritic cells may include, but are not limited to, long cytoplasmic processes or large cells with multiple fine dendrites. Phenotypic characteristics may include, but are not limited to, expression of one or more of MBC class I molecules, MHC class II molecules, CD1, CD4, CD11c, CD123, CDS8α, CD205 (Dec-205), 33D1, CD40, CD80, CD86, CD83, CD45, CMRF-44, CMRF-56, CD209 (DC-SIGN), CD208 (DC-LAMP), CD207 (Langerin) or CD206 (macrophage mannose receptor). Functional activity includes, but is not limited to, a stimulatory capacity for naive allogeneic T cells. Likewise, reference herein to “T-cell” should be read as including reference to cells which express one or more T-cell-type receptor and which carry out the one or more functions associated with cells generated in the thymus and to mutants or variants thereof. “Variants” include, but are not limited to, cells exhibiting some but not all of the morphological or phenotypic features or functional activities of DC and/or T-cells. “Mutants” include, but are not limited to, DC and/or T-cells which are transgenic wherein said transgenic cells are engineered to express one or more genes such as genes encoding antigens, immune modulating agents or cytokines or receptors. Reference herein to a DC and/or T-cells refers to both partially differentiated and fully differentiated DC and/or T-cells and to activated and non-activated. DC and/or T-cells.

Accordingly, a preferred embodiment of the present invention contemplates a method for modulating the immuno-activity of a DC and/or a T-cell, said method comprising contacting said DC and T-cell with an effective amount of an agent, which agent couples, binds or otherwise associates with a cell surface activation molecule, for a time and under conditions sufficient prevents, inhibits or otherwise down-regulates one or more functional activities of the said cells.

Preferably, the targeted DC is a myeloid DC. Preferably, the DC and/or T-cells are activated DC and/or T-cells.

A reference to an APC and/or lymphocyte being “immuno-active”, or other forms thereof such as “immuno-activity”, is a reference to a range of in vivo or in vitro activities of APC and/or lymphocyte, such as occurs in the context of an immune response. For example, immune activities contemplated herein include inter alia one or more of antigen endocytosis, antigen processing and/or presentation, as well as antigen detection or recognition or effecting the lysis of target cells displaying particular antigens. In the context of the present invention, a preferred APC is a DC and a preferred lymphocyte is a T-cell.

As detailed above, the range of immuno-activities potentially displayed by an APC encompasses and includes, inter alia, antigen endocytosis, processing and presentation, on contact with an agent capable of eliciting such a response. Similarly, the range of immuno-activities potentially displayed by a lymphocyte encompasses and includes, inter alia, activation of macrophages, stimulation of B-cells to produce antibody and causing the lysis of particular target cells displaying recognised antigens. The modulation of such “immuno-activity”, therefore, refers to the ability to alter, suppress or increase, up- or down-regulate or otherwise affect the level and/or amount of APC and/or lymphocyte immuno-activity. Preferably, the modulation results in suppression, inhibition or down-regulation of APC and/or lymphocyte immuno-activity. In this context, modulating a cell's immuno-activity also encompasses and includes affecting the viability of the said cell or cells and in a preferred embodiment, extends to their depletion, inactivation and/or eventual apoptosis.

The method of the present invention is performed by contacting an APC and/or lymphocyte, and preferably a DC and/or a T-lymphocyte, with an “agent”, through which one or more functional activities of said APC and/or lymphocyte is prevented, inhibited or otherwise down-regulated. As mentioned, the down-regulation may be as a result of inactivation of one or more activities of the said cells and/or by depletion or lysis of said APC and/or lymphocyte.

Preferably the APC and/or lymphocytes are activated DC and/or T-lymphoblasts.

Reference herein to an “agent” should be understood as a reference to any proteinaceous or non-proteinaceous molecule which couples, binds or otherwise associates with the subject cell-surface activation molecule. The subject agent may be linked, bound or otherwise associated with any proteinaceous or non-proteinaceous molecule. For example, it may be associated with a molecule which permits targeting to a localized region. Said proteinaceous molecule may be derived from natural, recombinant or synthetic sources including fusion proteins or following, for example, natural product screening. Said non-proteinaceous molecule may be derived from natural sources such as, for example, natural product screening or may be chemically synthesized, or may be derived from high throughput screening of chemical libraries. Suitable agents that may have applicability in the instant invention include, for example, any protein comprising one or more immunoglobulin domains, and extend to antibodies within the immunoglobulin family of plasma proteins which includes immunoglobulin (Ig)A, IgM, IgG, IgD and IgE. The term “antibody” includes and encompasses fragments of an antibody such as, for example, a diabody, derived from an antibody by proteolytic digestion or by other means including but not limited to chemical cleavage. An antibody may be a “polyclonal antibody” or a “monoclonal antibody”. “Monoclonal antibodies” are antibodies produced by a single clone of antibody-producing cells. Polyclonal antibodies, by contrast, are derived from multiple clones of diverse specificity. The term “antibody” also encompasses hybrid antibodies, fusion antibodies and antigen-binding portions, as well as other antigen-binding proteins such as T-associated binding molecules.

The agent of the present invention may form a complex with a cell-surface activation molecule on an APC and/or lymphocyte, by binding or otherwise associating with the said molecule via any suitable interactive bonding mechanism including, for example, non-covalent bonding such as ionic bonding or co-valent bonding. In a preferred embodiment, the cell-surface activation molecule is bound by an amount of antibody effective to form a complex under conditions which result in the prevention, inhibition or down-regulation of one or more functional activities of an APC and/or lymphocyte and, in particular, a DC and/or T-lymphocyte. An “effective amount” means an amount necessary to at least partly obtain the desired response, viz to prevent, inhibit or down-regulate one or more functional activities of an APC and/or lymphocyte, or to increase or otherwise potentiate the onset of an appropriate inhibitory or down-regulatory response, or to induce or otherwise effect the depletion, lysis or malfunctioning of an APC and/or lymphocyte.

By “cell-surface activation molecule” is meant a molecule the expression of which is up-regulated upon stimulation of an APC and/or lymphocyte. For example, a DC may be activated upon exposure to a foreign antigen to which the generation of an immune response is desirable. Similarly, a T-cell may be activated in response to exposure to an antigen presented to it by a DC. Furthermore, DC and/or T-cells may be activated in other circumstances, such as where aberrant activation occurs in response to their exposure to a “self” molecule, thereby leading to the induction of an undesirable auto-immune response.

Accordingly, in a preferred embodiment of this aspect of the present invention, the agent comprises a monoclonal antibody (mAb) such as, for example, against CD83, or a derivative, fragment, homolog, analog or chemical equivalent or mimetic of the antibody and the cell-surface activation molecule extends to encompass derivatives, fragments, homologs, analogs or chemical equivalents or mimetics of the cell-surface activation molecule expressed on the surface of a DC and/or a T-cell.

Preferably, the DC is a myeloid DC. In one embodiment, the T-cell is a CD4⁺ CD8⁻ T-cell, and in another embodiment, the T-cell is a CD4⁻CD8⁺ T-cell.

“Derivatives” include fragments, parts, portions, mutants, variants and mimetics from natural, synthetic or recombinant sources including fusion proteins. Parts or fragments include, for example, active regions of an agent or cell-surface activation molecule. Derivatives may be derived from insertion, deletion or substitution of amino acids. Amino acid insertional derivatives include amino and/or carboxylic terminal fusions as well as intra-sequence insertions of single or multiple amino acids. Insertional amino acid sequence variants are those in which one or more amino acid residues are introduced into a predetermined site in the protein although random insertion is also possible with suitable screening of the resulting product. Deletion variants are characterized by the removal of one or more amino acids from the sequence. Substitutional amino acid variants are those in which at least one residue in the sequence has been removed and a different residue inserted in its place. An example of substitutional amino acid variants is conservative amino acid substitution. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences including fusions with other peptides, polypeptides or proteins.

Chemical and functional equivalents of the agent or cell-surface activation molecule should be understood as molecules exhibiting any one or more of the functional activities of these molecules and may be derived from any source such as by being chemically synthesized or identified via screening processes such as natural product screening.

The derivatives of an agent or cell-surface activation molecule include fragments having particular epitopes or parts of the entire molecule fused to peptides, polypeptides or other proteinaceous or non-proteinaceous molecules.

Analogs of an agent or cell-surface activation molecule contemplated herein include, but are not limited to, modification to side chains, incorporating of unnatural amino acids and/or their derivatives during peptide, polypeptide or protein synthesis and the use of cross-linkers and other methods which impose conformational constraints on the proteinaceous molecules or their analogs.

Examples of side chain modifications contemplated by the present invention include modifications of amino groups such as by reductive alkylation by reaction with an aldehyde followed by reduction with NaBH₄; amdination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of mixed disulphides with other thiol compounds; reaction with maleimide, maleic anhydride or other substituted maleimide; formation of mercurial derivatives using 4-chloro-mercuribenzoate, 4-chloromercuriphenylsulphonic acid, phenylmercury chloride, 2-chloro-mercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, oinithine, sarcosine, 4-amino-3-hydroxy-6-methylheptanoic acid, 2-thienyl alanine and/or D-isomers of amino acids. A list of unnatural amino acid contemplated herein is shown in Table 1.

TABLE 1

| Non-conventional amino acid | Code |
|---|---|
| α-aminobutyric acid | Abu |
| α-amino-α-methylbutyrate | Mgabu |
| aminocyclopropanecarboxylate | Cpro |
| aminoisobutyric acid | Aib |
| aminonorbornylcarboxylate | Norb |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl)carbamylmethyl)glycine | Nnbhm |
| 1-carboxy-1-(2,2-diphenylethylamino)cyclopropane | Nmbc |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-Nmethylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Cross-linkers can be used, for example, to stabilize 3D conformations, using homobifunctional cross-linkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

To effectively prevent, inhibit or otherwise down-regulate an immuno-activity of an APC and/or lymphocyte, by binding or associating with a cell-surface activation molecule, a range of approaches and conditions may be utilized. For example, an agent may be conjugated with another molecule. Such an agent-conjugate may comprise an antibody as hereinbefore described, linked via means such as chemical linkage, to another molecule such as but not limited to a peptide, polypeptide, protein, enzyme, nucleic acid molecule including an oligonucleotide, carbohydrate molecule or a polysaccharide molecule or radioactive atom. Antibody conjugates may in some circumstances, be more efficacious in causing the desired outcome. For example, an antibody may be conjugated with a toxic component so as to induce cellular inactivation and/or lysis upon (i.e. during or after) the formation of an antibody/cell-surface activation molecule complex on the surface of an APC and/or lymphocyte. Methods for the conjugation of molecules such as, but not limited to, toxic molecules are well known in the art. In this embodiment of the invention, such antibody conjugates may directly induce inactivation and/or lysis of an APC and/or lymphocyte.

To the extent that the agent is an antibody, an APC and/or lymphocyte may undergo opsonization by the antibody thereby leading to the induction of one or more effector mechanisms, including lysis of opsonized DC and/or T-lymphocytes by killer cells such as, but not limited to, NK and K cells, which express an Fc receptor and/or uptake of opsonized DC and/or T-lymphocytes by phagocytic cells (such as macrophages), which also express an Fc receptor.

Without wishing to limit the present invention to one theory or mode of action, it is proposed that CD83 antibody—bound to the surface of DCs and/or T-cells—interacts with Fc receptors on the surface of, inter alia, NK cells, leading to the release of granules, which cause the destruction of the opsonized target DC and/or T-cells. This process is known in the art as antibody-dependent cell-mediated cytotoxicity (ADCC).

Any conditions sufficient to result in the prevention, inhibition or down-regulation of one or more functional activities of an APC and/or a lymphocyte are suitable for the practice of the present invention. In yet another alternative, an agent of the present invention, in particular an antibody, may activate the complement system, triggering a complement-mediated lytic response. Complement-mediated cytotoxicity or lysis is particularly suited to immuno-therapeutic applications where the depletion, down-regulation or destruction of specific cells is desirable. Where an agent such as an Ab is engaged by the complement system, chemical conjugation with toxic moieties becomes unnecessary. A very localized immune response, culminating in cell lysis, may result. Under most conditions, lysis is substantially restricted to the cell to which the agent binds and occurs without the necessity to conjugate a toxic moiety, the presence of which may increase the risk that cells other that target cells are concomitantly inadvertently affected.

In all instances, cytotoxicity requires that an agent recognizes and binds, complexes or otherwise associates with a cell-surface activation molecule. Preferably the agent comprises an antibody to CD83.

Without wishing to limit the invention to any one mode of action or practice, the particular nature of the effector mechanism which is stimulated may determine the nature of the immuno-activity which is modulated as well as the type and extent of modulation effected. For example, an antibody conjugated with a highly toxic component may induce rapid lysis of an APC and/or a lymphocyte once bound to a targeted cell-surface activation molecule. Lysis may proceed directly, and cellular debris may be removed by, for example, circulating macrophages. An antibody coupled to a less toxic molecule may, for example, have the effect of inhibiting the metabolic activity of an APC, causing it to be less able to process and present, or less efficient in processing and presenting, antigen. Similarly, the capability of a lymphocyte to detect and distinguish antigens from different types of cells may be inhibited. Alternatively, cell-mediated cytotoxicity may result in, for example, the ability of a lymphocyte to activate macrophages or to stimulate a B-cell to produce antibody, or of an APC to endocytose antigen, being disrupted or prevented. Or it may cause the number of APC and/or lymphocyte to be depleted, or result in the interruption of APC and/or lymphocyte differentiation and/or activation. ADCC may eventually be expected to result in the death (lysis and removal) of targeted cells including, in the context of the present invention, DC and/or T-cells.

Accordingly, depending on the particular conditions under which an agent such as a mAb associates with a cell-surface activation molecule, a functional activity of the said APC and/or lymphocyte may be affected. Preferably, the functional immuno-activity which is modulated is one or more of antigen endocytosis, antigen processing and/or presentation in the case of APC, and activation of macrophages, stimulation of the production of antibodies by B-cells, and/or killing of target cells, in the case of lymphocytes, the modulation being elicited on contact of an antibody and/or an antibody-conjugate with an antigen.

In one embodiment of the present invention, modulation of immuno-activity of an APC and/or lymphocyte is achieved via a mAb and, in particular, a mAb against CD83, and inter alia ADCC. Preferably the APC is an activated DC and the lymphocyte is an activated T-lymphoblast.

Accordingly, the present invention in a preferred embodiment provides a method for modulating the immuno-activity of an APC and/or lymphocyte, said method comprising contacting said APC and/or lymphocyte with an effective amount of a mAb for a time and under conditions sufficient to prevent, inhibit or otherwise down-regulate one or more of antigen endocytosis, antigen processing and/or antigen presentation by said APC and activation of macrophages, stimulation of antibody production, and/or killing of target cells by said lymphocyte.

Preferably said monoclonal antibody is against CD83.

Still more preferably, the APC is a DC and the lymphocyte is a T-lymphoblast.

The method of the present invention is therapeutically beneficial in circumstances where inactivation of APC and lymphocyte functional activity and, in particular, DC and T-cell functional activity may be desirable. Such circumstances include those wherein an unwanted, aberrant or otherwise undesirable immune response is or has been elicited. An example is in procedures involving allogeneic grafts such as bone marrow transplantation and tissue and/or organ transplantation, where graft versus host and/or host versus graft incompatibility may result in host cell or transplant cell rejection, respectively. An “allogeneic graft” is a graft wherein the donor is of the same species as the recipient, but is MHC (or minor histocompatibility antigen) incompatible. In graft versus host incompatibility, effector cells of an immuno-competent allograft stimulated by host or donor APC presenting host antigen may target host cells. Alternatively, in host versus graft incompatibility, antigens derived from the allograft may be endocytosed, processed and presented by host or donor DC to effector cells of the host's immune system, as hereinbefore described. Recipient T-lymphocytes are activated to target donor histocompatibility antigens. In each case, residual T-lymphocytes may activate and contribute to donor T-lymphocyte—recipient T-lymphocyte reactivity. In any case, the immune response comprises immuno-activity which directly or indirectly contributes to transplant and/or host tissue rejection.

The population of DC and/or T-cells which are treated in accordance with the methods of the present invention may be located in vivo or in vitro and may comprise activated or differentiated DC and/or T-cells. Generally, but not necessarily, activation of DC and T-cells is concomitant with further cellular differentiation and also proliferation in the case of T-cells.

The agent of the present invention may, in one embodiment, be administered to a subject. Alternatively, DC and T-cells isolated from a subject may be specifically destroyed or otherwise inactivated or rendered non-functional by contacting said cells in vitro with an effective amount of an agent, which agent couples, binds or otherwise associates with a cell-surface activation molecule, for a time and under conditions sufficient to prevent, inhibit or otherwise down-regulate one or more functional activities of said cells.

Preferably, the population of DC and T-cells is within a subject.

Accordingly another aspect of the present invention is directed to a method for modulating an immune response in a subject, said method comprising administering to said subject an effective amount of an agent, which agent couples, binds or otherwise associates with an antigen presenting cell's and/or lymphocyte's surface activation molecule for a time and under conditions sufficient to prevent, inhibit or otherwise down-regulate one or more functional activities of said APC and/or lymphocyte.

Preferably the APC is a DC and the lymphocyte is a T-cell.

Reference herein to cells of an “immuno-competent” allograft should be understood as a reference to a population of allograft cells which comprises immune cells. By “immune cells” is meant cells which directly or indirectly contribute to one or more aspects of an immune response, such as facilitating antigen presentation, phagocytosis, immune effector mechanisms, antibody dependent cytotoxicity, antibody production and cytokine production, inter alia, as hereinbefore defined.

Examples of immuno-competent allografts include bone marrow cells and spleen cells. Highly immature cells such as stem cells, which retain the capacity to differentiate into a range of immune or non-immune cell types, should also be understood to satisfy the definition of “immune cells” as utilized herein, due to their capacity to differentiate into immune cells under appropriate conditions. Accordingly, an allograft comprising stem cells is also an immuno-competent graft within the scope of the present invention. It should further be understood that, in the context of the present invention, an immuno-competent graft may also comprise a non-immune cell component. This would be expected where an unpurified bone marrow or spleen cell graft, for example, is the subject of transplantation, since such a graft may be expected to comprise red blood cells, fibroblasts, platelets, adipocytes and other such non-immune cells.

It should be understood that the allograft that is transplanted into a host may be in any suitable form. For example, the graft may comprise a population of cells existing as a single cell suspension or it may comprise a tissue sample fragment or an organ. The allograft may be provided by any suitable donor source. For example, the cells may be isolated from an individual or from an existing cell line. The tissue allograft may also be derived from an in vitro source such as a tissue sample or organ, which has been generated or synthesized in vitro.

A “subject” in the context of the present invention includes and encompasses mammals such as humans, primates (gorillas, marmosets, macaques) and livestock animals (e.g. sheep, pigs, cattle, horses, donkeys); laboratory test animals (e.g mice, rabbits, rats and guinea pigs; and companion animals (e.g. dogs and cats). Preferably, the mammal is a human.

A reduction in the presentation of an allograft antigen to host T cells or host antigen to donor T cells, as processed and presented by DC, has the potential to prevent or limit the extent of an immune response. This reduction in presentation may be achieved by, for example either down-regulation of antigen-processing or reducing or preventing antigen presentation. A reduction in the number or efficacy of host lymphocytes responding to graft antigen, or of graft lymphocytes responding to host antigen has the potential to prevent or limit the extent of an immune response. This reduction in number or efficacy of host lymphocytes may be achieved by, for example, complement or ADCC mediated lysis of responding lymphocytes or by inhibition of one or more functions of responding lymphocytes. In this context, a “host” is synonymous with “subject” and includes a human subject, as well as other animals such as other mammals inter alia, as hereinbefore described.

Accordingly, another aspect of the present invention provides a method for down-regulating the immuno-activity of an immuno-competent graft, said method comprising administering to said subject an effective amount of an agent, which agent couples, binds or otherwise associates with an APC's and/or a lymphocyte's surface activation molecule, for a time and under conditions sufficient to prevent, inhibit or otherwise down-regulate one or more functional activities of said APC and/or a lymphocyte.

Agents suitable for use in this aspect of the present invention include antibodies and, more particularly, monoclonal antibodies, as hereinbefore described. Preferably the mAb is against CD83. Preferably the subject is a human.

In a most preferred embodiment of the present invention, an agent comprising a mAb against CD83 or an appropriate functional derivative, homolog, analog, chemical equivalent or mimetic thereof, may be administered to a human subject undergoing or have undergone allogeneic graft transplantation, such as bone marrow transplantation, in the expectation that the said mAb may locate, bind or otherwise associate with a cell-surface activation molecule of a donor or graft antigen-presenting DC and/or a donor or graft lymphocyte and hence down-regulate its function, thereby ameliorating or preventing the development of graft versus host disease or graft rejection.

Hence the methods of the present invention have application in the treatment and/or prophylaxis of conditions characterized by aberrant, unwanted or otherwise inappropriate immuno-activity of an allogeneic immuno-competent craft such as occurs in graft verses host disease. The incidence of graft versus host disease may be observed in any situation where an allogeneic immuno-competent graft is required to be transplanted into a host recipient, such as pursuant to treatment for certain forms of cancer wherein bone marrow. transplants are necessitated.

Accordingly, in a preferred embodiment, the present invention provides a method for down-regulating the immuno-activity of a bone marrow graft in a subject, said method comprising administering to said subject an effective amount of mAb against CD83, for a time and under conditions sufficient to prevent, inhibit or otherwise down-regulate one or more functional activities of a DC and/or T-cell.

Reference to “down-regulating” the immuno-activity of an immuno-competent graft should be understood as a reference to at least partially down-regulating said activity. Without wishing to limit the present invention to any one theory or mode of action, it will be understood that down-regulation may be brought about under a range of different conditions. These include, for example, the utilization of an antibody-conjugate, the assistance of cells involved in cell-mediated cytotoxicity, ADCC and/or the involvement of the complement-mediated processes, as described hereinbefore, and the extent of down-regulation will be influenced by the nature of the conditions, inter alia.

In this context, an “effective amount” means an amount necessary to at least partly obtain the desired response, or to delay the onset or inhibit progression or halt altogether the onset or progression of a particular condition being treated. The amount varies depending upon the health and physical condition of the subject being treated, the taxonomic group of the subject being treated, the degree of protection desired, the formulation of the composition, the assessment of the medical situation and other relevant factors. It is expected that the amount will fall in a relatively broad range, which may be determined through routine trials.

Accordingly, another aspect of the present invention contemplates a method for the prophylactic and/or therapeutic treatment of a condition characterized by the aberrant, unwanted or otherwise inappropriate immuno-activity of an immuno-competent graft, said method comprising contacting said graft with an effective amount of an agent or a derivative, homolog, analog, chemical equivalent or mimetic thereof, which agent couples, binds or otherwise associates with an APC's and/or a lymphocyte's surface activation molecule, for a time and under conditions sufficient to prevent, inhibit or otherwise down-regulate the immuno-activity of said APC and/or lymphocyte.

Preferably the immuno-competent graft comprises allogeneic bone marrow cells.

Preferably the APC is a DC, the lymphocyte is a CD4⁺ CD8⁻ T-lymphoblast and the agent comprises the mAb against CD83.

More particularly, the present invention contemplates a method for the prophylactic and/or therapeutic treatment of a condition characterized by the aberrant, unwanted or otherwise inappropriate immuno-activity of an immuno-competent graft, in a subject, said method comprising contacting said graft with an effective amount of an agent or a derivative, homolog, analog, chemical equivalent or mimetic thereof, which agent couples, binds or otherwise associates with an APC's and/or a lymphocyte's surface activation molecule derived from said graft, for a time and under conditions sufficient to prevent, inhibit or otherwise down-regulate the said inappropriate immuno-activity of said graft.

Preferably, the said subject is a human. Preferably, the said condition is graft versus host disease.

Still more preferably said graft is an allogeneic bone marrow graft, spleen cell graft or a stem cell graft.

Reference herein to “therapeutic” and “prophylactic” treatment is to be considered in its broadest context. The term “therapeutic” does not necessarily imply that a subject is treated until total recovery. Similarly, “prophylactic” does not necessarily mean that the subject will not eventually contract a disease condition. Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

The methods of the present invention may have further use in the prophylactic and/or therapeutic treatment of a range of other conditions characterized by an unwanted or undesirable immune response. Such conditions include, inter alia, those wherein the response is inappropriate as well as those wherein the response may be regarded as being physiologically normal but is nevertheless undesirable. These often involve the presence of activated DC or T-lymphocytes. Examples include auto-immune conditions, chronic inflammatory conditions, asthma and hypersensitivity, allergies to innocuous agents and transplant rejection.

More particularly, conditions which are proposed to be treatable using the methods of the present invention encompass auto-immune and inflammatory disorders such as, for example, rheumatoid arthritis, lupus erythematosus, systeric lupus erythematosus, Hashimotos thyroiditis, multiple sclerosis, myasthenia gravis, type 1 diabetes, auto-immune anaemia, thrombocytopenia, inflammatory bowel disease and Crohn's disease.

In any condition, where undesirable responses are triggered by the presentation of antigen, the methods of the present invention may find useful application.

Accordingly, another aspect of the present invention contemplates a method for the prophylactic and/or therapeutic treatment of a condition characterized by an aberrant, unwanted or otherwise inappropriate immune response in a subject, said method comprising administering to said subject an effective amount of an agent, which agent couples, binds or otherwise associates with an APC's and/or a lymphocyte's surface activation molecule, for a time and under conditions sufficient to prevent, inhibit or otherwise down-regulate the immuno-activity of said APC and/or lymphocyte.

The present invention further extends to pharmaceutical compositions and formulations comprising the said agents for use in conjunction with the instant methods. Such pharmaceutical compositions and formulations may be administered to a human or animal subject in any one of a number of conventional dosage forms and by any one of a number of convenient means. The agent of the pharmaceutical composition is contemplated to exhibit therapeutic activity when administered in an amount which depends on the particular case. The variation depends, for example, on the human or animal and the agent chosen. A broad range of doses may be applicable. Considering a patient, for example, from about 0.1 mg to about 1 mg of agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The agent may be administered in a convenient manner such as by the oral, intravenous (where water soluble), intraperitoneal, intramuscular, subcutaneous, intradermal or suppository routes or implanting (e.g. using slow release molecules). The agent may be administered in the form of pharmaceutically acceptable non-toxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in accordance with the present invention may be co-administered with one or more other compounds or molecules. By “co-administered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of micro-organisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of micro-organisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

The present invention further contemplates a combination of therapies, such as the administration to a subject of the agent of the present invention in a pharmaceutical composition or formulation together with a low dose of immuno-suppressive drugs.

Yet another aspect of the present invention is directed to the use of an agent of the present invention in the manufacture of a pharmaceutical composition or formulation for use in the method of the invention.

The present invention is further described by the following non-limiting Examples.

### EXAMPLE 1

### Material and Methods

Preparation of CD83-Fusion Protein

CD83-Ig, consisting of the extra-cytoplasmic segment of human CD83 fused at the C-terminus to human IgG1-F_{c}, was synthesized and purified from transfected COS-7 cell conditioned medium as previously described (Hock et al., 2001, supra).

Aniti-CD83

Rabbit polyclonal anti-CD83 serum was prepared by immunization with CD83 fusion proteins, as described (Hock et al., 2001, supra). The IgG fraction was purified from this serum, and from non-immunized rabbit serum (HiTrap Protein A, Amersham Pharmacia Biotech, Sydney). Anti-human IgG, anti-mouse serum protein, and anti-foetal calf serum protein activity was removed from both IgG fractions by passage through columns of immobilized human IgG (Intragam, CSL Ltd, Parkville, Vic.), mouse serum, and foetal calf serum protein (HiTrap NHS-activated, Amersham). The final preparations, designated RA83 and RAneg, respectively, consisted of a single major protein band of 150 kD (non-reducing SDS-PAGE electrophoresis) with minor contaminants. On reduction, only two bands were visible, 25 and 50 kD, corresponding to IgG light and heavy chains. RA83, but not RAneg, bound to the CD83⁺ Hodgkins lymphoma derived cell line LA28, as shown by flow cytometry after secondary staining with FITC-goat anti-rabbit Ig (Dako). RA83, but not RAneg, also bound to CD83-Ig or soluble native CD83 antigen captured by the anti-CD83 mAb Hb15a immobilized on ELISA plates (see below).

F_{ab} fragments of RA83 and RAneg were generated by papain digestion (Harlow and Lane, Anitibodies: A Laboratory Manual. Cold Spring Harbor Laboratory, New York, 1988). The reactions were stopped with iodoacetamide and the digests were dialysed in PBS and passed through a HiTrap Protein-A column to remove unreacted IgG and F_{c} fragments. Unbound protein consisted of a major band at ˜42 kD (=F_{ab}) and several lighter bands at 20-30 kD (non-reducing SDS-PAGE). F_{ab} derived from whole RA83, but not from RAneg, also stained LA28 cells.

Soluble CD83 ELISA

Ninety-six well ELISA plates (Maxisorb, Nunc, Roskilde, Denmark) were coated with 75 μl of the CD83 mAb Hb15a (Immunotech, Marseille, France) at 1 μg/ml in 0.1 mol/l sodium carbonate at pH 9.6 by overnight incubation at 4° C., and were blocked with bovine serum albumin (Sigma, protease free, 20 mg/ml. 1.5 hr at room temperature). Standards (CD83-Ig fusion protein) and samples for analysis (culture supernatants) were diluted 1:5 or 1:10 v/v in 5% v/v FCS in PBS and 0.05% v/v Tween-20 (PBST) and assayed in triplicate, 75 μl/well. After a 2-hr incubation at room temperature, the wells were washed and 75 μl of RA83 or RAneg at 5 μg/ml, in huIgG (5 mg/ml) and 5% FCS in PBST was added. After 1 hr, the wells were washed again and anti-rabbit-peroxidase conjugate was added (diluted 1:5000 in 5% FCS, 75 μl/well, Jackson ImmunoResearch Laboratories, West Grove Pa., USA). After a further 1-hr incubation and wash, bound peroxidase was detected with tetramethyl benzidine (Sigma) and H₂O₂. Mean negative control (RAneg) absorbances (450 nm) were subtracted from corresponding RA83 values for each standard and sample. sCD83 was expressed in pg/ml after correction for the difference in MW between CD83-Ig and the calculated MW from the published sequence.

Preparation of Cells

In some experiments MoDC were compared with CD11c⁺ blood dendritic cells (IC). Blood DC were prepared, as described (MacDonald et al., Blood, in press, 2002), from buffy coats provided by the Australian Red Cross. Briefly, PBMC were isolated on Ficoll-Hypaque Plus (Amersham Pharmacia Biotech, Sweden), and lineage positive cells were removed by immuno-magnetic depletion with a cocktail of mAbs (CD3, 11b, 14, 16, 19) and anti-mouse IgG-coated magnetic beads (Polysciences, Pennsylvania, USA). The remaining cells were treated with Vitalyse (BioErgonomics, Minnesota, USA) to remove residual erythrocytes, stained with FITC-sheep anti-mouse immunoglobulin antibody (FITC-SAM, Silenus, Melbourne) and either PECy5-HBLA-DR or PE-CD11c, as required, and sorted for FITC-negative/dim and PECy5 or PE bright events from the forward and side scatter live gate (Vantage, Becton Dickinson. San Jose, Calif.). When cultured alone, these sorted blood DC were placed in 10% w/v FCS in RPMI1640 with added glutamine and antibiotics (=“medium”) and added IL-3 (10 ng/ml, Gibco BRL, Grand Island, N.Y.) and GM-CSF (200 U/ml, Sandoz-Pharma, Auckland, NZ) (Kohrgruber et al., J Immunol. 163:3250, 1999).

MoDC were prepared from PBMC after depletion of CD2⁺ cells by rosetting with neuraminidase treated sheep red blood cell. The rosette negative cells (ER⁻), generally 50-60% CD14⁺, were cultured at 0.5×10⁶ CD14⁺ cells/ml in medium containing GM-CSF (200 U/ml) and IL4 (50 U/ml, Sigma) (Vuckovic et al., Exp Hematol 26:1255, 1998). Conversion of monocytes to iMoDC was checked after five days by staining with CD1a (Na134), FITC-SAM and CD14-PE. Maturation/activation was induced by addition of lipo-polysaccharide (LPS; 1 μg/ml, Sigma). For some experiments, PBS-washed MoDC were fixed in 2% paraformaldehyde (PFA) for 20 min at ambient temperature, washed in PBS, then in medium twice, incubated in medium overnight at 37° C., and washed again.

Rosette positive cells (ER⁺) were 80-90% CD3⁺ and were utilized as a source of T-cells either in this form or were further purified by immuno-magnetic depletion after staining with mAbs for CD11b, CD 14, CD16, CD19, and HLA-DR. These further purified T-cells were >95% CD3⁺. For some experiments, T-cells and NK-cells were purified by sorting FITC⁻, PE⁻ and PE⁺ events in the live gate after staining ER⁺ preparations with CD14-, 19-, 34-, HLA-DR-FITC and CD56-PE.

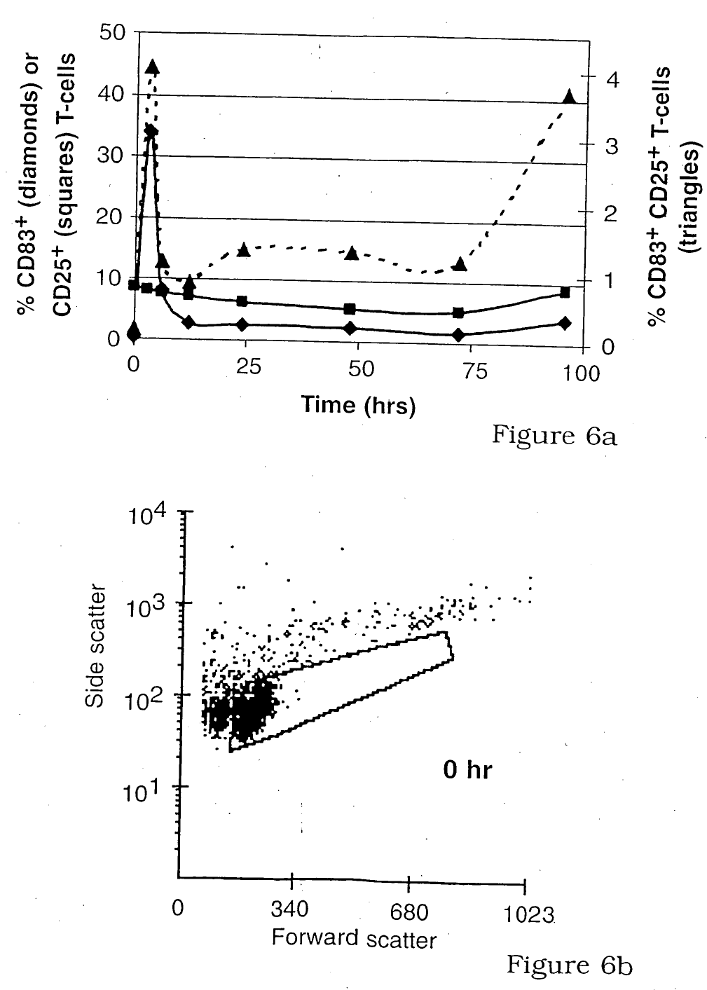

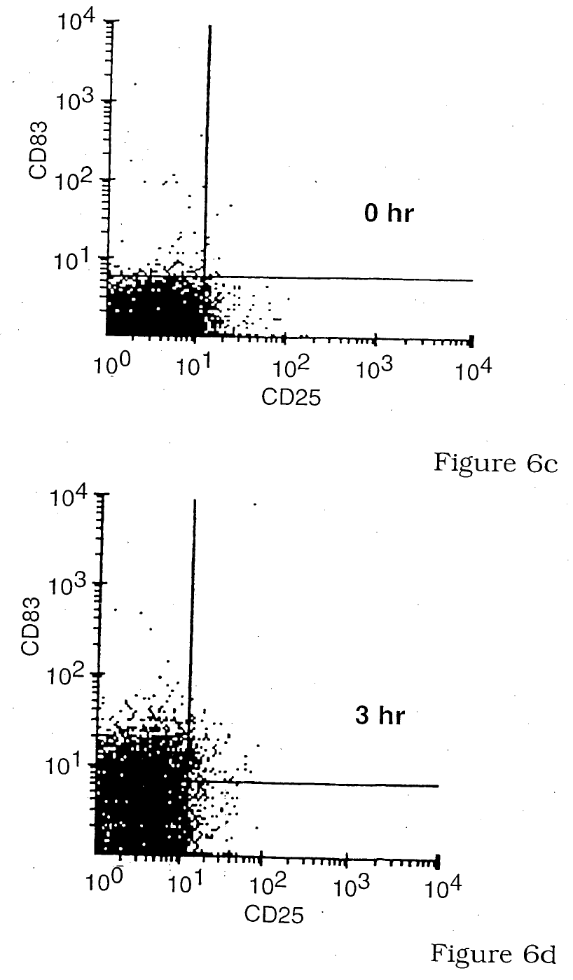

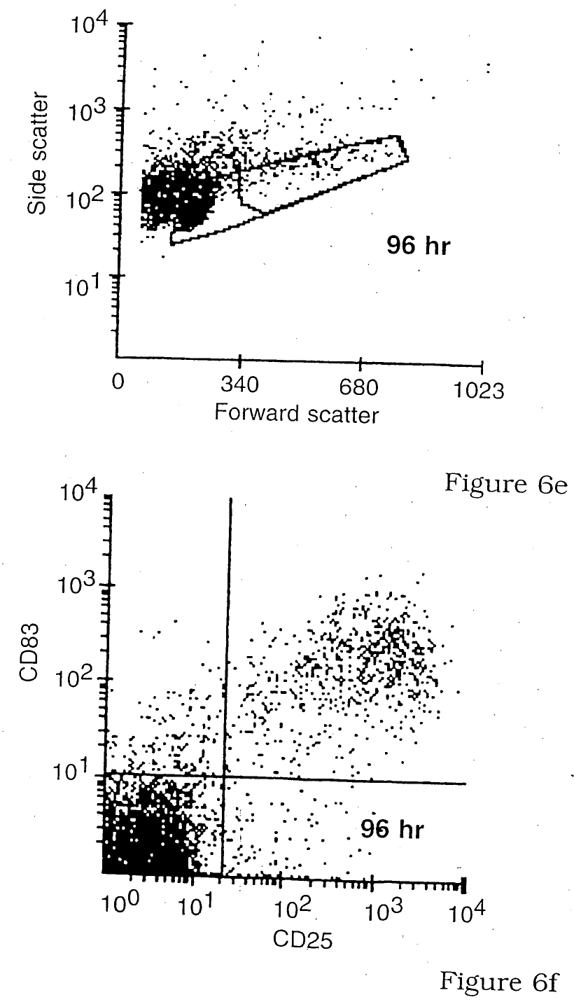

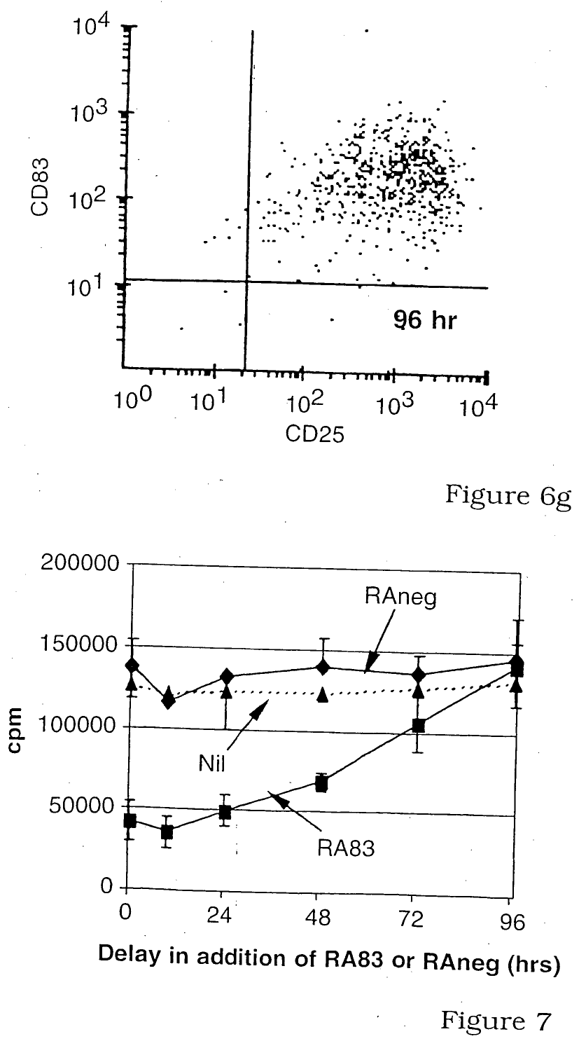

For the ⁵¹Cr release assay, NK-cells were sort purified from the normal lymphoid gate by negative selection (FITC⁻, PE⁻) from a 65 hr mixed leucocyte reaction (MLR) consisting of R⁺ cells and allogeneic iMoDC (20:1), after staining with CD3-PE, CD14-, CD19-, D34-, HLA-DR-FITC. T-cell blasts (PE⁺) were sort purified simultaneously from the T-cell blast gate (see FIG. 6E).

Staining and Flow Cytometry

Cells were stained with antibodies by incubation for 20 min on ice, washed with 2% w/v FCS in PBS containing 0.05% NaN₃, and resuspended in 1% PFA to await flow cytometry (FACSCalibur, BD, Cellquest acquisition software). The following commercial antibodies were employed for staining cells: CD83-FITC, CD83-PE and purified CD83 (Hb15a, Immunotech), CD11c-PE, CD25-PE (BD), CD86-FITC and CD86-PE (Pharmingen). Staining with unconjugated mAbs was detected, as indicated, either with FITC-SAM, or with biotinylated anti-mouse Ig (Sigma) followed by streptavidin-PECy5 (Dako). Intracellular staining with CD83-FITC and Ki67-FITC (Dako) was effected with “Fix & Perm” (Caltag, Burlingame, Calif., USA).

Lymphocyte Stimulation

The one way mixed leucocyte reaction (MLR) was done in 96-well U-bottomed culture plates with up to 5000 MoDC or blood DC per well and 10⁵ allogeneic ER⁺ or purified T-cells per well. DC were pre-incubated for 10 min at 37° C. in the wells with RA83, RAneg or medium alone prior to the addition of T-cells. The MLR plate was cultured in a 37° C./5%CO₂ incubator for 4 days, pulsed with 1 μCi ³H-thymidine (Amersham, Sydney, NSW) per well, incubated for a further 16 hr, harvested (Tomtec Mach 3M, Connecticut, USA), and counted (Trilux 1450, Wallac, Finland). Mean counts per minute (cpm)±SE, for replicate wells, are reported without subtraction of counts for stimulators (DC) or responders alone.

T-cells were also stimulated by culture in U-bottomed 96-well plates pre-coated with purified CD3 mAb OKT3 and the co-stimulatory mAb CD28 (Leu28, BD) in PBS. After blocking and washing with medium, either RA83 or RAneg (final concentrations 5 μg/ml) or medium alone were added, along with 10⁵ T-cells, to give 200 μl/well. The plates were incubated for 4 days, pulsed, harvested and counted as for the MLR.

For the ⁵¹Cr release assay, ≦10⁶ sort purified T-cell blasts were loaded with 0.1 mCi ⁵¹Cr-NaCrO₄ (Amersham), washed, resuspended in the MLR conditioned medium and dispensed into 96-well conical culture plates at 2,500 blasts/well, with either RA83 or RAneg at 5 μg/ml. The sort purified NK-cells, also resuspended in MLR conditioned medium, were added at up to a 20-fold excess. Wells were made up to 175 μl, cultured for 4 hr, and 25 μl of supernatant was mixed with 150 μl scintillant (Optiphase Supermix Wallac) for counting (Trilux 1450).

### EXAMPLE 2

### CD83 Expression by DC

To clarify the role of CD83 in DC-T-cell interactions, CD83 expression by DC was characterized and compared with CD86 expression. Because the production of soluble CD83 appeared to be a normal physiological process (Hock et al., 2001, supra), the effects of soluble CD83 (CD83-Ig) and polyclonal anti-CD83 (RA83) on DC induced T-cell responses were investigated.

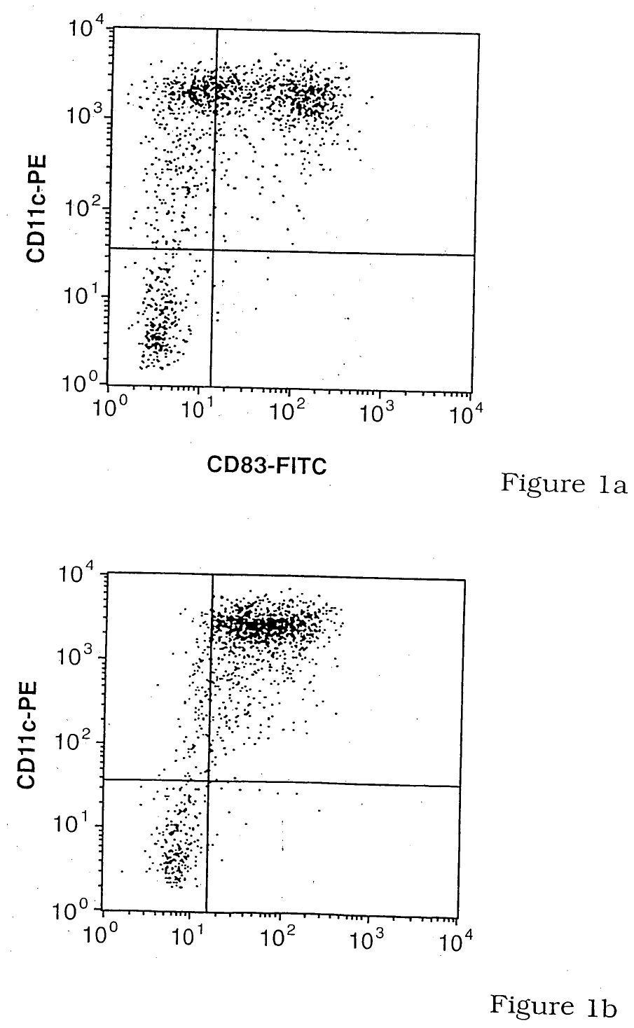

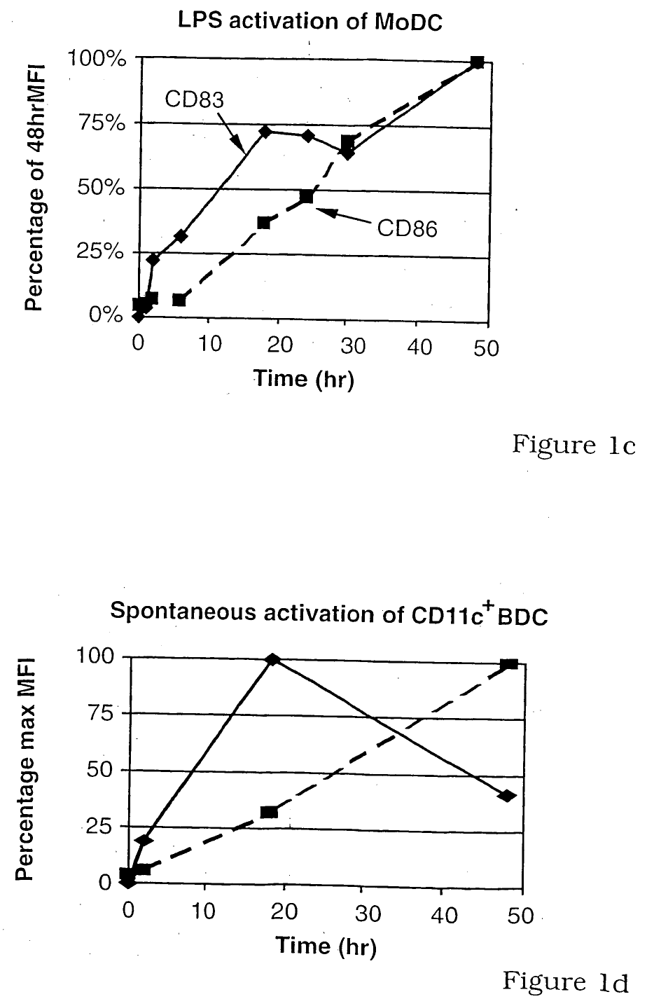

CD83 expression by CD11c⁺ (myeloid) blood DC was compared and contrasted with that for the supposed in vitro, homologue, monocyte-derived-DC (MoDC). A significant minority of CD11c⁺ blood DC failed to spontaneously up-regulate CD83 when cultured in GM-CSF, IL-3 and 10% w/v FCS, whereas virtually all up-regulated CD86 (FIGS. 1A and 1B). Immature MoDC (iMoDC) do not spontaneously up-regulate CD83 during their preparation in GM-CSF, IL-4 and 10% w/v FCS, but all iMoDC became CD83⁺ and CD86⁺⁺ after lipo-polysaccharide (LPS) addition. For both types of DC, significant levels of surface CD83, but not CD86, appeared within 2 hr of the activation/maturation stimulus (FIGS. 1C and 1D). The mean fluorescence intensities continued to increase for 18 hr, after which MoDC CD83 plateaued briefly and then continued to rise, whereas blood DC CD83 levels fell. In contrast, CD86 continued to increase in both types of DC (FIGS. 1C and 1D).

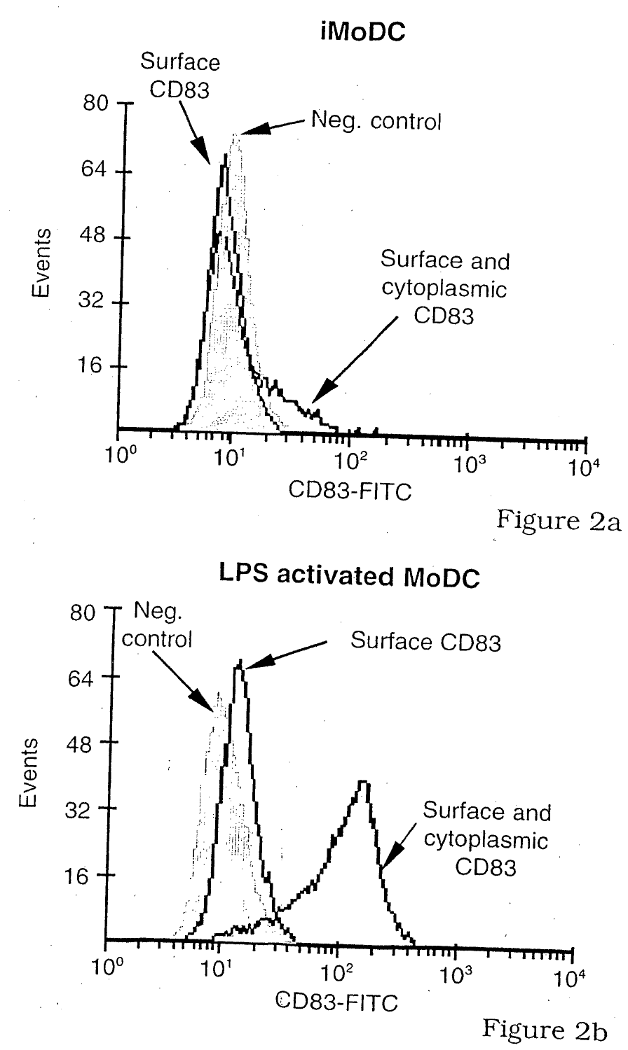

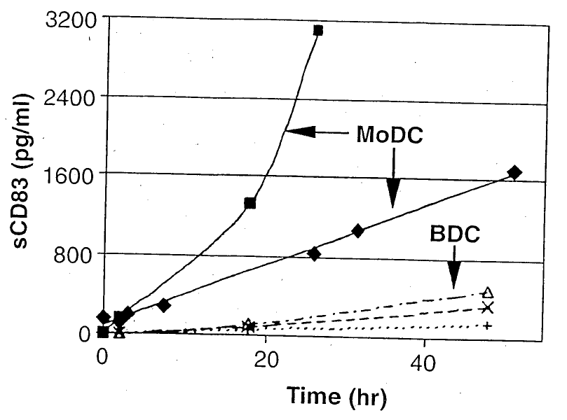

Data shown in FIG. 2A confirm that iMoDC have minimal surface CD83 and low but detectable levels of cytoplasmic CD83. Cytoplasmic CD83 in fresh blood DC was, however, not detectable. Cytoplasmic, CD83 was increased considerably more than surface CD83 in LPS activated MoDC, as can be seen in FIG. 2B. Both types of DC secreted sCD83 into the medium, but MoDC secreted much greater quantities when stimulated with LPS compared to spontaneously maturing blood DC (FIG. 2C).

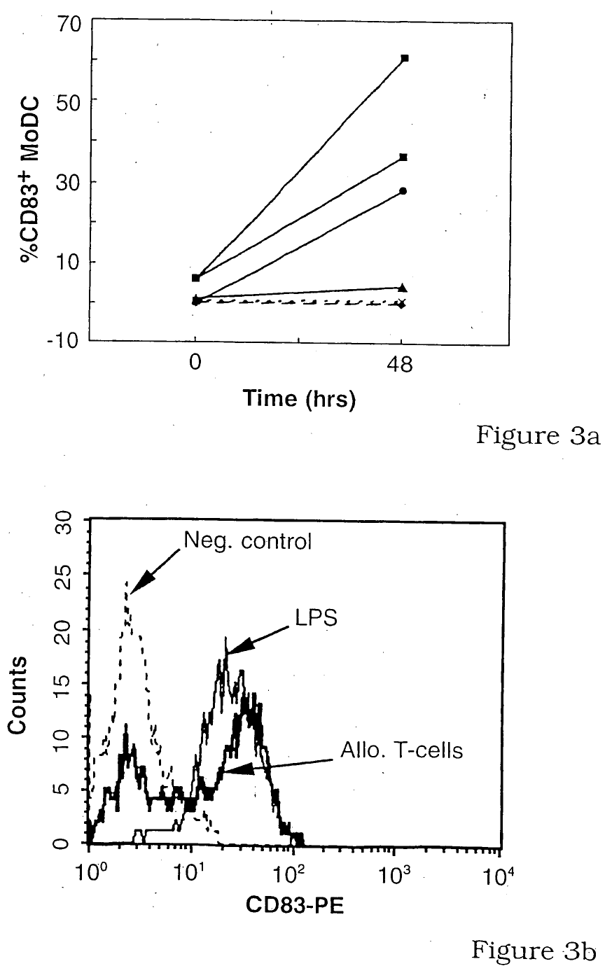

CD40L is expressed on activated T-cells. However, co-culture of iMoDC with freshly isolated allogeneic T-cells did not consistently up-regulate surface CD83 on the MoDC (FIG. 3A), even though T-cells had become activated since a proliferative response was consistently observed. On those occasions when CD83 was up-regulated, the MoDCs became divided into discrete CD83⁺ and CD83⁻ populations, which contrasted with the unimodal expression observed with LPS activation (FIG. 3B). The MoDC were at least as effective as fresh blood DC in inducing proliferation of allogeneic T-cells.

### EXAMPLE 3

### Functional Effects of Anti-CD83 and CD83-Ig

To investigate the potential contribution of CD83 to DC-T-lymphocyte interactions, purified rabbit polyclonal IgG anti-CD83 (RA83) was used. First, the findings of Armitage et al. (1996, supra), that RA83 blocks the proliferative response of PBMC to tetanus toxoid (TT), were confirmed. Furthermore, RA83 blocked the proliferative response of ER⁺ to allogeneic blood DC and to allogeneic MoDC (see below).

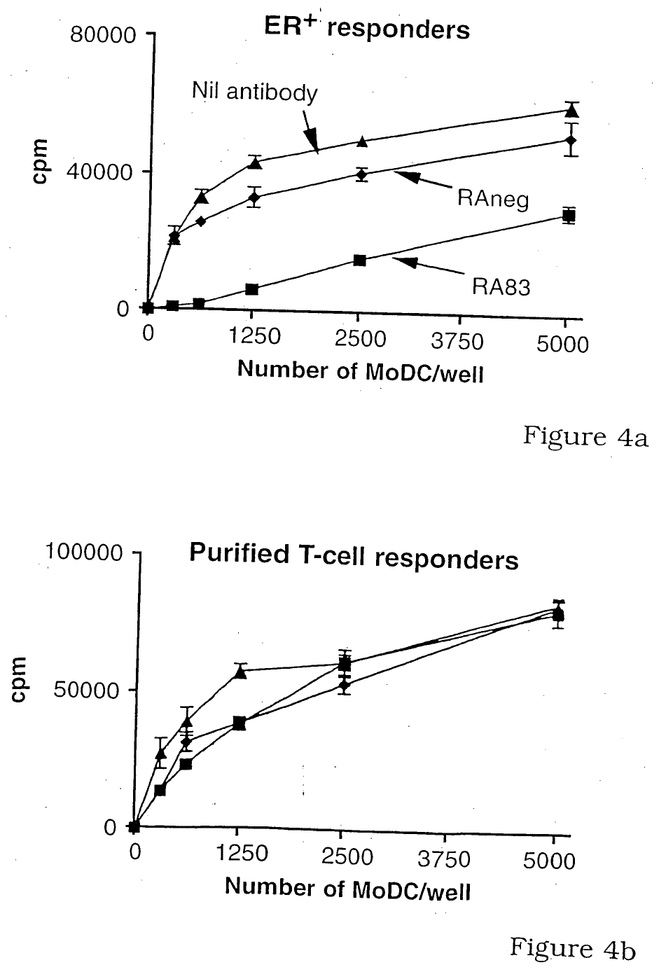

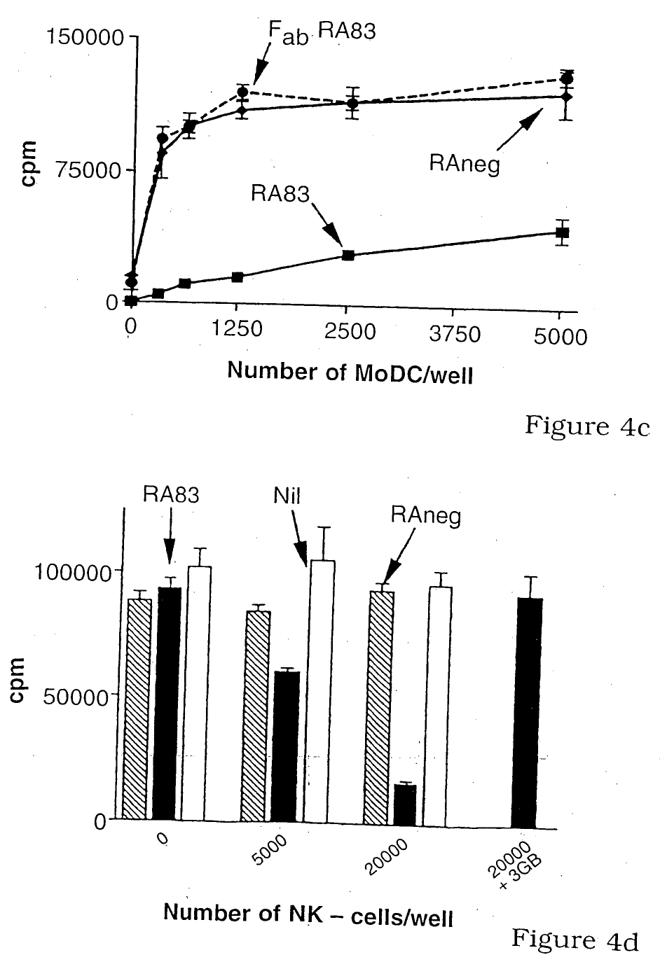

In subsequent experiments MoDC were used as stimulators. However, blockade was abrogated if the ER⁺ responders were further purified by immuno-magnetic depletion with a cocktail of mAbs for CD11b, CD14, CD16, CD19 and HLA-DR (see FIGS. 4A and 4B).

On culturing MoDC with allogeneic ER⁺, it was found that the degree of blockade of ³H-thymidine incorporation was RA83 dose dependent and rarely achieved 100% (donor variable). The effect was shown to be specific for CD83 because it could be overcome by the addition of CD83-Ig, but not human IgG, to the MLR. CD83-Ig added alone did not significantly affect the T-cell proliferative response in the MLR.

### EXAMPLE 4

### RA83 Blockade is Due to NK-Cell Mediated ADCC of CD83⁺ Targets

Further depletion experiments with each mAb alone, from the above cocktail, suggested that the blockade was mediated by CD16⁺ NK cells which co-purified with T-cells in the rosetting procedure. The possibility of an antibody-dependent cellular cytotoxicity (ADCC) mechanism for the blockade was suggested by the failure of blockade with either (i) Fab fragments of the RA83 antibody (FIG. 4C), or with (ii) the CD16 function blocking mAb 3G8 (FIG. 4D) (Perussia and Trinchieri, J Immunol 132:1410, 1984). This mechanism was confirmed by the addition of purified CD56⁺ NK-cells to wells containing sort purified T-cells, allogeneic MoDC and RA83, but not RAneg (FIG. 4D). It was therefore concluded that, contrary to previous proposals, RA83 did not inhibit the MLR by blocking a functional interaction between CD83 and its ligand.

### EXAMPLE 5

### A Target of RA83 ADCC is in the Responder Cell Preparation

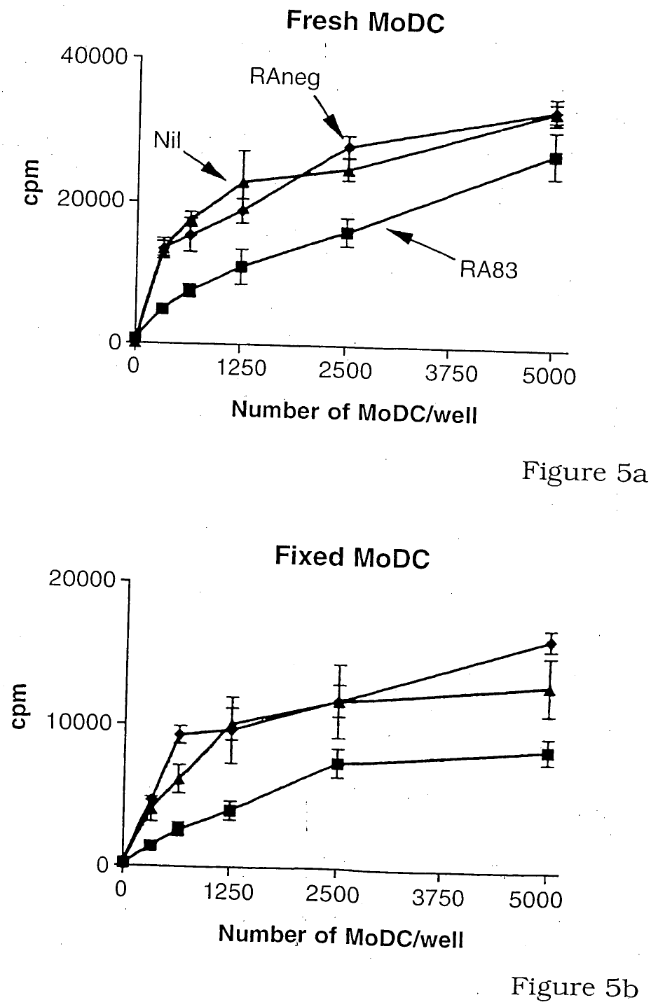

The next test focussed on ascertaining whether RA83 inhibited the MLR when the allogeneic iMoDC used as stimulators were prevented from up-regulating surface CD83 or secreting sCD83. This was done by fixation of the iMoDC in paraformaldehyde prior to culture with ER⁺ responders. The percentage blockade of ³H incorporation due to RA83, relative to RAneg, for fixed iMoDC was equal to that for unfixed MoDC, even though the absolute counts were approximately halved (FIGS. 5A and 5B). From these data it was concluded that NK-cell mediated lysis of CD83⁺ MoDC targets does not account for the RA83 blockade, and that the ADCC target cell accounting for the observed reduction in proliferation must be in the responder preparation.

DC are not the only possible candidate CD83⁺ target for ADCC. B-cells can also express CD83 (Kozlow et al., 1993, supra) and these were present as minor contaminants in the ER⁺ responder preparations used above. However, they are not functionally important in RA83 blockade of the MLR, because-immuno-magnetic depletion of CD19⁺ cells, or of HLA-DR⁺ cells, from ER⁺ responder preparations had no effect on blockade.

### EXAMPLE 6

### CD83 Expression by T-Cells

Given the above findings, the expression of CD83 on T-lymphocytes in co-cultures of ER⁺ and MoDC was investigated. Low levels of CD83 were rapidly induced on a high proportion of CD3⁺ T-cells, reaching a maximum in 3 hr and then decaying back to near-background levels at 12 hr (FIGS. 6A and 6D). Further experiments showed that this 3-hr induction of CD83 expression also occurred when the stimulator cells were omitted from the culture. The 3-hr staining was specific for CD83 because it was blocked by pre-incubation of the Hb15a CD83 staining nab with CD83-Ig fusion protein, but not with human IgG. Also, the polyclonal RA83, but not RAneg, positively stained these cells at 3 hr.

ER⁺ cultured with allogeneic MoDC for longer periods resulted in the appearance of small but variable numbers of CD83+, CD25⁺ T-cells. At 96 hr, a subset of CD83⁺, CD25⁺, CD3⁺ cells was clearly evident (FIG. 6F). This subset included virtually all of the T-cell blasts, judged from the high forward scatter characteristics (see FIG. 6G), but on some occasions CD83⁺, CD25⁺ T-cells were also found in the normal lymphoid gate. Further phenotyping revealed that the majority of CD83⁺, CD25⁺ T-cells were CD4⁺, CD8⁻ and stained positively for the intracellular proliferation marker Ki67. Hence, this CD83 expressing subset consisted of proliferating T-cells in the MLR.

### EXAMPLE 7

### RA83 Blocks the MLR by NK-Cell Mediated ADCC Lysis of CD83⁺ T-Cell Blasts

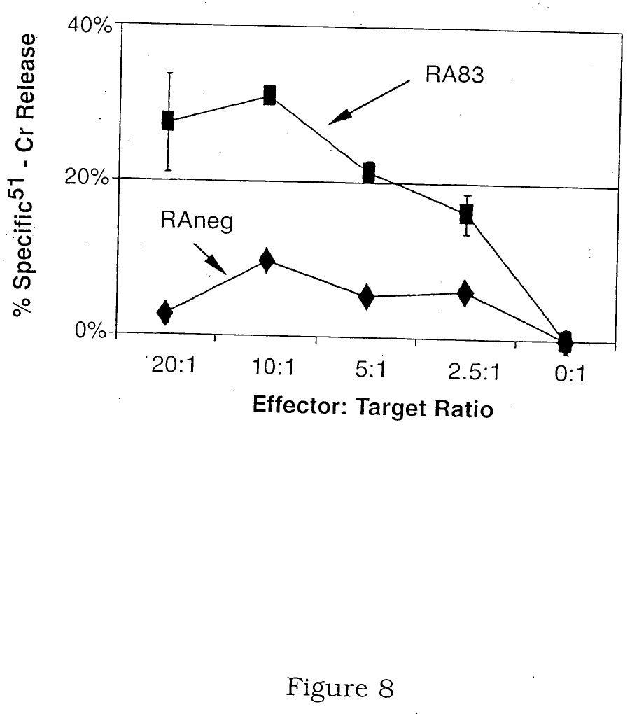

Whether RA83-mediated blockade of T-cell proliferation in the allogeneic MLR was caused, at least in part, by lysis of CD83⁺ T-cells by autologous NK-cells was then investigated. A 24-hr delay in addition of RA83 did not abrogate blockade (FIG. 7). It was, therefore, concluded that the 3-hr CD83⁺ T-cells were not targets, at this time, in RA83-mediated blockade of the MLR. The data in FIG. 7 suggested that the majority of targets of interest appeared more than 24 hr after initiating the MLR. NK-cells and T-cell blasts from a 65-hr co-culture of ER⁺ and allogeneic iMoDC were therefore sort-purified. In a 4-hr ⁵¹Cr-release assay, the T-cell blasts were lysed by the NK-cells in the presence of RA83 but only minimally in the presence of RAneg (FIG. 8).

In conclusion, then, human T-lymphocytes expressed CD83 in a highly regulated fashion, and RA83-dependent blockade of the MLR was due to NK-cell mediated ADCC lysis of responding CD83⁺ T-cell blasts.

### EXAMPLE 8

### RA83 Effects NK-Cell Mediated ADCC Lysis of CD83⁺ DC

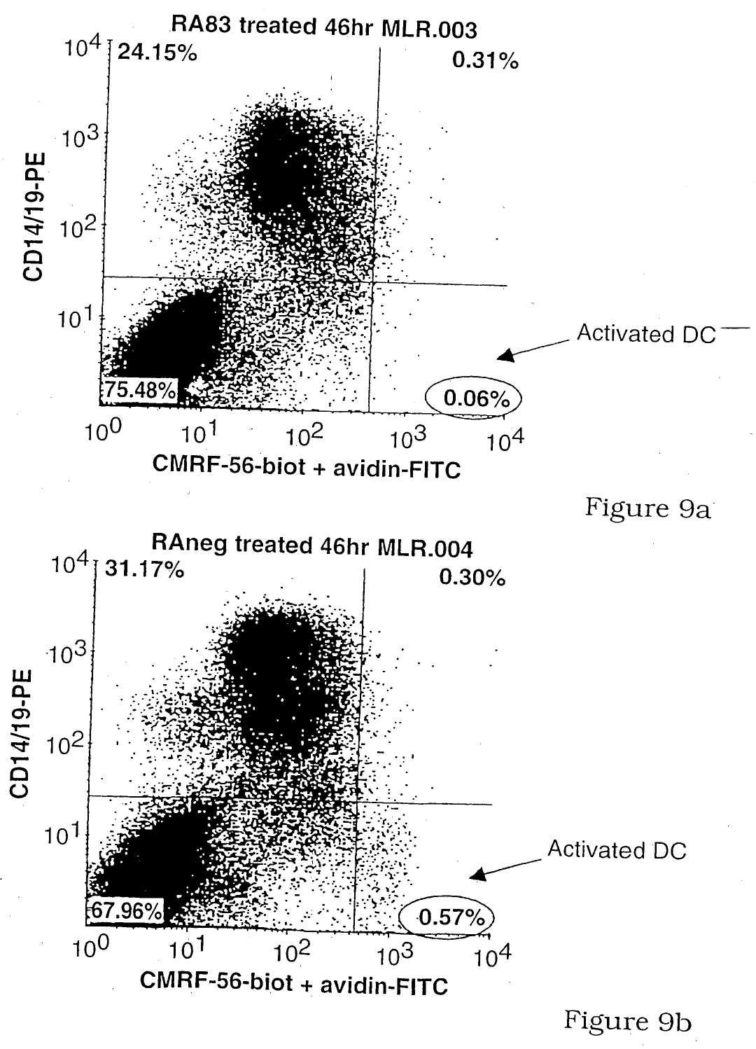

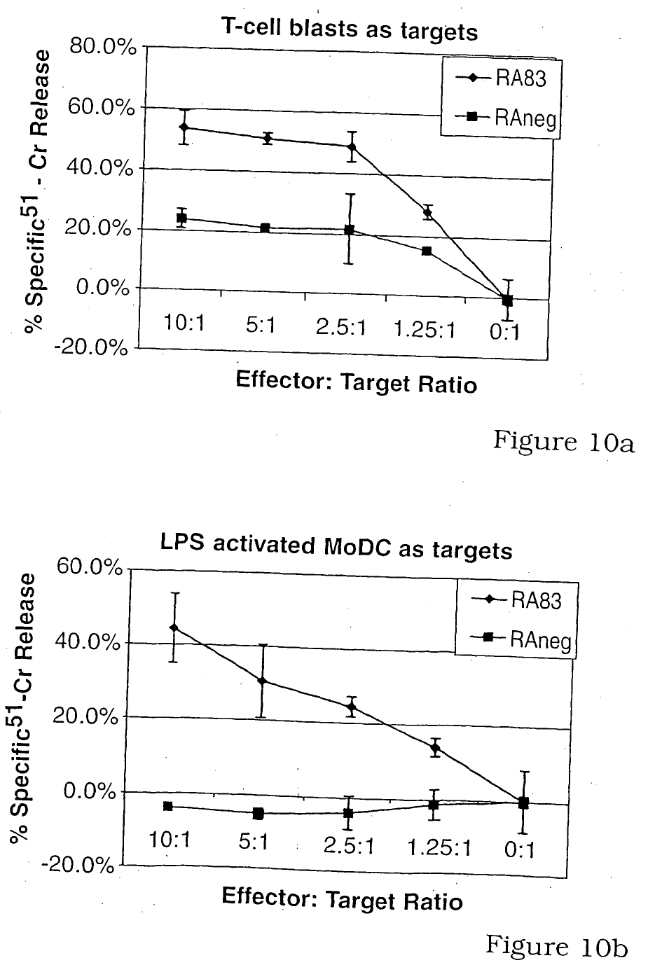

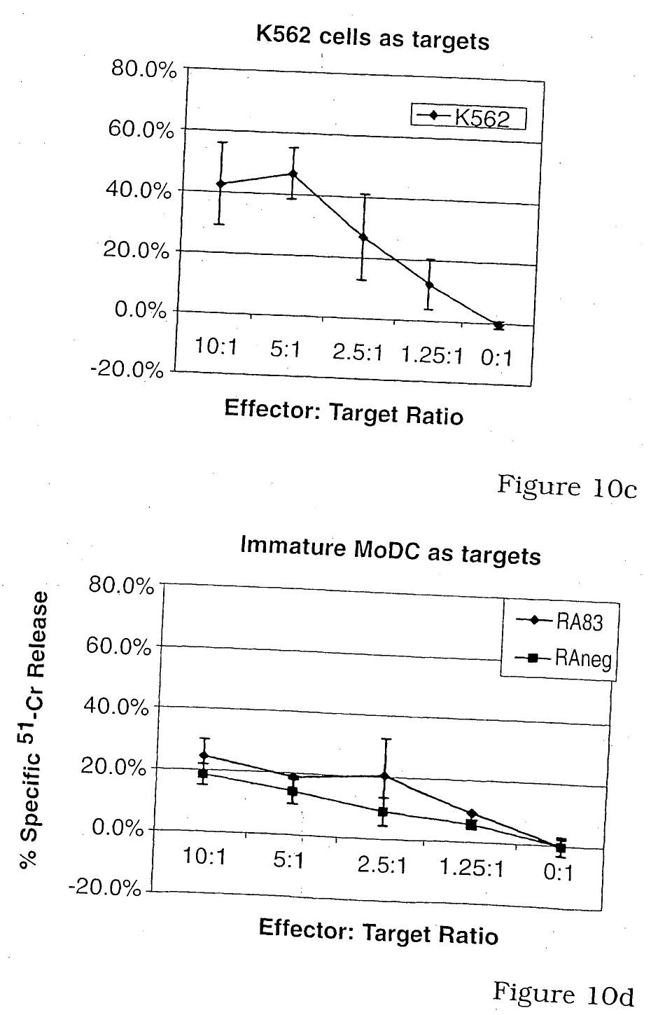

Data shown in FIGS. 9A and 9B provide evidence that RA83 also depletes activated DC in the MLR. Activated blood DC (CMFR-56⁺, CD14/19⁻ cells) in PBMC from two donors, co-cultured for 46 hr were 89% depleted in the presence of CD83Ab (RA83), compared with the control. Furthermore, activated MoDC are depleted by RA83 + NK-cells (FIG. 10).

Those skilled in the art will appreciate that the present invention described herein is susceptible to variations and modifications other than those specifically described. It is to be understood that the present invention includes all such variations and modifications. The present invention also includes all of the steps, features, compositions and compounds referred to or indicated in this specification, individually or collectively, and any and all combinations of any two or more of said steps or features.

### BIBLIOGRAPHY

Armitage et al., In Leucocyte Typing VI. T. Kishimoto, ed. Garland Publishing. Inc, New York, p. 593, 1996a.
Armitage et al., In Leucocyte Typing VI. T. Kishimoto, ed. Garland Publishing Inc, New York, p. 595, 1996b.
Cramer et al., Int Immunol 12:1347, 2000.
Fujimoto et al., Cell 108:755, 2002.
Harlow and Lane. Anitibodies: a laboratory manual. Cold Spring Harbor Laboratory, New York, 1988.
Hock et al., Int Immunol 13:959, 2001.
Kohrgruber et al., J Immunol 163:3250, 1999.
Kozlow et al., Blood 81:454, 1993.
Lechmann et al., J Exp Med 194:1813, 2001.
MacDonald et al., Blood, in press, 2002.
Perussia and Trinchieri, J Immunol 132:1410, 1984.
Scholler et al., J Immunol 166:3865, 2001.
Sorg et al., Pathology 29:294, 1997.
Vuckovic et al., Exp Hematol 26:1255, 1998.
Zhou et al., J Immunol 149:735, 1992.
Zhou et al., J Immunol 154:3821, 1995.

1. A method for modulating immuno-activity of a cell selected from a stimulator myeloid dendritic cell (DC) and a responder CD4⁺CD8⁻ T cell or CD4⁻CD8⁺ T cell said method comprising contacting said cell with an effective amount of an antibody or antigen-binding fragment thereof which couples, binds or otherwise associates with CD83 and in turn prevents, inhibits or otherwise down-regulates one or more functional activities of said cell by inducing cell lysis.

2. The method of claim 1 wherein the antibody is a monoclonal antibody or a functional equivalent thereof.

3. The method of claim 1 wherein the cell is mammalian derived.

4. The method of claim 3 wherein the mammalian cell is a human cell.

5. The method of claim 2 wherein the functional equivalent is an antigen-binding derivative, fragment, homolog, analog or chemical equivalent of the antibody.

6. The method of claim 1 wherein lysis is caused by antibody-dependent cell-mediated cytotoxicity.

7. The method of claim 2 wherein the antibody in conjugated with a toxic component which induces or otherwise facilitates lysis of the APC and/or lymphocyte.

8. A method for modulating the immuno-activity of a myeloid DC and/or T cell, said method comprising contacting said DC and/or T cell with an effective amount of a monoclonal antibody specific for CD83 for a time and under conditions sufficient to prevent, inhibit or otherwise down-regulate one or more of antigen endocytosis, antigen processing and/or antigen presentation by said DC and activation of macrophages, stimulation of antibody production, and/or killing of target cells by said T cell.

9. A method for modulating an immune response in a subject, said method comprising administering to said subject an effective amount of an antibody which couples, binds or otherwise associates with a myeloid DC's and/or T cell's surface activation molecule for a time and under conditions sufficient to induce cell lysis of said DC and/or T cell.

10. A method for down-regulating the immuno-activity of an immuno-competent graft, said method comprising administering to said subject an effective amount of an antibody which couples, binds or otherwise associates with a myeloid DC's and/or a T cell's CD83 molecule, for a time and under conditions sufficient to induce cell lysis of said DC and/or a T cell.

11. A method for down-regulating the immuno-activity of a bone marrow graft in a subject, said method comprising administering to said subject an effective amount of monoclonal antibody against CD83, for a time and under conditions sufficient to prevent, inhibit or otherwise down-regulate one or more functional activities of a DC and/or T-cell by inducing cell lysis.

12. A method for the prophylactic and/or therapeutic treatment of a condition characterized by the aberrant, unwanted or otherwise inappropriate immuno-activity of an immuno-competent graft, said method comprising contacting said graft with an effective amount of an antibody or a derivative, homolog, analog, chemical equivalent or mimetic thereof, which couples, binds or otherwise associates with a myeloid DC's and/or a T cell's surface activation CD83 molecule, for a time and under conditions sufficient to prevent, inhibit or otherwise down-regulate the immuno-activity of said DC and/or T cell by inducing cell lysis.

13. The method of claim 17 wherein the immuno-competent graft comprises allogenic bone marrow cells.

14. The method of claim 12 wherein the lymphocyte is a CD4⁺ CD8⁻ or CD4⁻ CD8⁺ T cell.

15. A method for the prophylactic and/or therapeutic treatment of a condition characterized by the aberrant, unwanted or otherwise inappropriate immuno-activity of an immuno-competent graft in a subject, said method comprising contacting said graft with an effective amount of an antibody or a derivative, homolog, analog, chemical equivalent or mimetic thereof, which couples, binds or otherwise associates with a myeloid DC's and/or a T cell's CD83 molecule derived from said graft, for a time and under conditions sufficient to prevent, inhibit or otherwise down-regulate the said inappropriate immuno-activity of said graft by inducing cell lysis.

16. The method of claim 15 wherein the subject is a mammal.

17. The method of claim 16 wherein the mammal is a human.

18. The method of claim 15 wherein the condition is graft versus host disease.

19. The method of claim 15 wherein the graft is an allergenic bone marrow graft, spleen cell graft or stem cell graft.

20. A method for the prophylactic and/or therapeutic treatment of a condition characterized by an aberrant, unwanted or otherwise inappropriate immune response in a subject, said method comprising administering to said subject an effective amount of an antibody or antigen-binding fragment thereof which couples, binds or otherwise associates with CD83 on the surface of a myeloid DC's and/or a CD4⁺CD8⁻ T cell or CD4⁻CD8⁺ T cell, for a time and under conditions sufficient to prevent, inhibit or otherwise down-regulate the immuno-activity of said DC and/or T cell by inducing cell lysis.
